(19) United States
(12) Patent Application Publication
(10) Pub. No.: US 2019/0071463 A1
(43) Pub. Date: Mar. 7, 2019

(54) METHOD FOR PREPARING A MIXTURE OF MONOSACCHARIDES AND/OR OF OLIGOSACCHARIDES AND/OR OF POLYSACCHARIDES VIA PURIFICATION OF A HYDROLYSATE OF LIGNOCELLULOSIC MATERIALS

(71) Applicant: INSTITUT POLYTECHNIQUE DE GRENOBLE, Grenoble (FR)

(72) Inventors: Christine CHIRAT, Genoble (FR); Dominique LACHENAL, Echirolles (FR); Jeremy BOUCHER, Saint Gaudens (FR)

(21) Appl. No.: 16/080,167

(22) PCT Filed: Feb. 24, 2017
(86) PCT No.: PCT/FR2017/050415

§ 371 Date: Aug. 27, 2018

(30) Foreign Application Priority Data
Feb. 25, 2016 (FR) 1651569

(51) Int. Cl. C07H 1/08; C07H 3/02; C07H 3/06; C08B 37/00

(52) U.S. Cl. CPC C07H 1/08; C07H 3/02; C08B 37/0003; C08B 37/0057; C07H 3/06

(57) ABSTRACT

The reuse of the sugars from the by-products of the paper and cellulose industries and lignocellulosic biorefineries and facilitating the extraction and the purification of the sugars contained in the hydrolysates of wood. A method for preparing a mixture of monosaccharides and/or of oligosaccharides and/or of polysaccharides via purification of a hydrolysate of lignocellulosic materials, said hydrolysate comprising hemicelluloses in the form of monomers, of oligomers, and optionally of polymers. The method includes at least one step of oxidation of said hydrolysate with at least one oxidant. This method allows a mixture of monosaccharides and/or of oligosaccharides and/or of polysaccharides to be obtained having a reduced quantity of furfural and/or of hydroxymethylfurfural and comprising polymers having a reduced mass molecular in weight and/or in number.

### FIELD OF THE INVENTION

The field of this invention is that of the reuse of the sugars from the by-products of the paper and cellulose industries and lignocellulosic biorefineries.

In particular, the present invention relates to the preparation of a mixture of monosaccharides and/or of oligosaccharides and/or of polysaccharides via purification of a hydrolysate of lignocellulosic materials, said hydrolysate comprising hemicelluloses in the form of monomers, of oligomers, and optionally of polymers.

### TECHNOLOGICAL BACKGROUND

Lignocellulosic material is the main component of the cell wall of plants. It consists of cellulose, hemicelluloses, lignin and extractables in variable proportions according to the origin of the material. Wood represents most of the available lignocellulosic material.

The paper industry uses wood to extract the fibres of cellulose therefrom and thus produce paper pulp. According to a standard method, the cellulose is extracted from the wood via an alkaline treatment at high temperature (Kraft process). The products of degradation of the hemicelluloses, lignin and the other by-products obtained by this treatment are recovered in an effluent called black liquor. At present, this effluent is only used for energy purposes since its complexity does not make the extraction of the hemicelluloses possible, since the latter are for the most part degraded in the form of organic acids.

The use of the black liquor obtained for energy purposes is of interest since it allows paper industries to produce energy and to be energy-independent or even produce a surplus of energy. However, in the current environmental context, another reuse of the compounds, other than cellulose, contained in the wood is desirable.

The hemicelluloses of wood are small polymers (degree of polymerisation between 50 and 200) composed of various types of sugars, their composition varying according to their origin. The sugars present in the hemicelluloses include mannose, galactose, glucose, xylose and arabinose. Due to their composition, these hemicelluloses thus provide access to sugars other than glucose. These sugars are of particular interest since they are sugars from non-food raw materials, as opposed to the glucose obtained from starch (corn, wheat, potato).

Various methods have thus been developed to extract at least a portion of the hemicelluloses of the wood before carrying out the extraction of the fibres of cellulose via the Kraft process. The hemicelluloses are hydrolysed and obtained mainly in the form of monomers and oligomers. Until now, manufacturers that extracted the hemicelluloses from wood chips, were not interested in the reuse of the hemicelluloses, but only in the production of pure cellulose: to do this, they subject the wood chips to autohydrolysis involving the injection of vapour and treatment of the chips at 170-180° C. for 1 to 2 hours, which allows most of the hemicelluloses to be solubilised. Then, the chips and the solubilised hemicelluloses are subject to Kraft cooking, which allows fibres of cellulose to be produced, the hemicelluloses previously solubilised are in the black liquor, and are burned with the lignin (Herbert Sixta, Handbook of Pulp, Wiley-VCH, 2006, 4.2.7.1. Prehydrolysis, p. 325).

However, given the development of biosourced chemistry, the demand for sugars is growing, and it would be wise to recover the sugars produced from the hemicelluloses of the wood in order to reuse them, which would represent additional revenue for the factories that produce cellulose (Ragauskas A J, Nagy M, Kim D H, Eckert C A, Hallett J P, Liotta C L, From wood to fuels-integrating biofuels and pulp production Industrial Biotechnology 2006; 2(1), 55-65).

The document US2015/0184260 A1 describes a method for producing fermentable sugars from a hydrolysate of wood comprising oligomers of hemicelluloses and from oligomers of glucose extracted from cellulose via enzymes. The sugars obtained can thus be used to produce biofuel or biochemical products.

The document EP 2067793 A1 describes the use of a hydrolysate of wood, obtained from a hydrothermal treatment of wood chips, for the production of biodegradable and renewable polymers and of various products such as films or gels.

However, it remains difficult to extract and purify the sugars contained in the hydrolysates of wood obtained by hydrolysis treatment. Indeed, these hydrolysates contain, besides the sugars of the hemicelluloses in the form of monomers and oligomers, products of degradation of the sugars such as furfural and hydroxymethylfurfural, precipitated and dissolved lignin, certain extractables of the wood, polymerised compounds (which can come from reactions between the lignin and furans, or even between the lignin and extractables, or from the polymerisation of the furans with each other) and organic acids.

The presence of these various compounds poses problems on several levels for the rest of the treatment of the hydrolysates. The presence of lignin and/or of other polymerised compounds (other than polysaccharides or oligosaccharides) leads to sticky polymer deposits (DPC) on the equipment used, which requires a level of maintenance of the equipment that is too high and makes applying a method of treatment of the hydrolysates on the industrial scale difficult. There are also problems of purification of the sugars obtained because of the presence of the furfural and of hydroxymethylfurfural in the hydrolysate.

These problems are a real obstacle to the chemical reuse of the sugars from the hemicelluloses and to the industrialisation of the methods of extraction and of treatment of these sugars.

### GOALS

In this context, the present invention aims to achieve at least one of the following goals.

One of the essential goals of the invention is to provide a new method for purification of a hydrolysate of lignocellulosic materials.

Another essential goal of the invention is to provide a new method for purification of a hydrolysate of lignocellulosic materials without reducing the quantities of sugars present in this hydrolysate.

Another essential goal of the invention is to provide a new method that allows the quantity of furfural and/or of hydroxymethylfurfural contained in a hydrolysate of lignocellulosic materials to be reduced.

Another essential goal of the invention is to provide a new method that allows the mass molecular in weight and/or in number of the polymers, other than the hemicelluloses, contained in a hydrolysate of lignocellulosic materials to be reduced.

Another essential goal of the invention is to provide a mixture of monosaccharides and/or of oligosaccharides and/or of polysaccharides that can be easily purified.

Another essential goal of the invention is to provide a mixture of monosaccharides and/or of oligosaccharides and/or of polysaccharides having a reduced quantity of furfural and/or of hydroxymethylfurfural.

Another essential goal of the invention is to provide a mixture of monosaccharides and/or of oligosaccharides and/or of polysaccharides comprising polymers having a reduced mass molecular in weight and/or in number.

Another essential goal of the invention is to provide a mixture of monosaccharides and/or of oligosaccharides and/or of polysaccharides that poses fewer, or even no, problems of deposits on the equipment.

Another essential goal of the invention is to provide a mixture of monosaccharides and/or of oligosaccharides and/or of polysaccharides that poses fewer, or even no, problems of purification of the sugars.

### BRIEF DESCRIPTION OF THE INVENTION

These goals, among others, are achieved by the present invention which relates first of all to a method for preparing a mixture of monosaccharides and/or of oligosaccharides and/or of polysaccharides via purification of a hydrolysate of lignocellulosic materials, said hydrolysate comprising hemicelluloses in the form of monomers, of oligomers, and optionally of polymers, characterised in that the method comprises at least one step of oxidation of said hydrolysate with at least one oxidant.

Surprisingly, it was discovered that treating a hydrolysate of lignocellulosic materials with an oxidant allowed the mass molecular in weight and in number of the polymers precipitated or dissolved to be reduced and the quantity of furfural and of hydroxymethylfurfural (HMF) contained in the hydrolysate to be reduced without significantly reducing or deteriorating the quantities of sugars in the hydrolysate, as one might fear.

This method allows a mixture of monosaccharides and/or of oligosaccharides and/or of polysaccharides that can be used industrially and the sugars of which can be easily separated and purified to be obtained. The sugars from the hemicellulose can thus be reused without compromising the papermaking method of producing fibres of cellulose.

The invention also relates to a method for preparing a mixture of monosaccharides and/or of oligosaccharides and/or of polysaccharides, via purification of a hydrolysate of lignocellulosic materials resulting from a heat treatment of lignocellulosic materials in the presence of water or water vapour followed by a separation of the joint product consisting of a residue rich in cellulose and optionally in lignin, said hydrolysate comprising hemicelluloses in the form of monomers and of oligomers, and optionally of polymers, characterised in that the method comprises at least one step of oxidation of said hydrolysate with at least one oxidant.

Secondly, the invention relates to a mixture of monosaccharides and/or of oligosaccharides and/or of polysaccharides obtained via purification of a hydrolysate of lignocellulosic materials.

### Definitions

In the present disclosure, the singular indifferently refers to the singular and to the plural and vice versa, unless expressly indicated otherwise. The following definitions are given as examples for the interpretation of the present disclosure.

Lignocellulosic materials: they are the main component of the cell wall of plants and mainly consist of cellulose, hemicelluloses, lignin and extractables. These materials can come, inter alia, from wood, recycled wood, annual plants, agricultural residues (bagasse, straws, . . . ), or recovered papers and cardboards.

Hydrolysate of lignocellulosic materials: liquid phase obtained via heat treatment of lignocellulosic materials in the presence of water or water vapour. This treatment can be carried out only with water or water vapour, or also in an acid medium via the addition of a weak or strong acid, or in a slightly basic medium (Maki-Arvela P, Salmi T, Holmbom B, Willfor S and Yu D. Murzin, Synthesis of Sugars by Hydrolysis of Hemicelluloses—A Review, Chem. Rev. 2011, 111, 5638-5666, dx.doi.org/10.1021/cr2000042). It allows the separation of a hydrolysate and of a residue rich in cellulose and optionally in lignin. This treatment leads to the “Hydrolysate of lignocellulosic materials” and to a residue rich in cellulose and optionally in lignin. This residue can then be separated from the hydrolysate and used to manufacture paper pulp or cellulose for various uses (textile, chemical, or nanocrystals or microfibrils of cellulose for example).

Oligomers: in the context of the invention, “oligomers” means molecules comprising a number of monomer units between 2 and 25.

Polymers: in the context of the invention, “polymers” means molecules comprising a number of monomer units greater than 25.

Coloured compounds: in the context of the invention, “coloured compounds” means compounds that have an absorbance signal in absorption spectroscopy in the UV-visible range between 200 and 400 nm. These include, inter alia, polymers resulting from the products of degradation of the components of the wood.

### BRIEF DESCRIPTION OF THE DRAWINGS

FIG. 1 shows the effect of the treatments with hydrogen peroxide on the concentrations of furfural and hydroxymethylfurfural in the hydrolysate.

FIG. 2 shows the effect of the treatments with hydrogen peroxide on the concentrations of total C5 and C6 sugars (monomers+oligomers) in the hydrolysate.

FIG. 3 shows the effect of the treatment with ozone on the concentrations of furfural and hydroxymethylfurfural in the hydrolysate.

FIG. 4 shows the effect of the treatment with ozone on the concentrations of total C5 and C6 sugars (monomers+oligomers) in the hydrolysate.

### DETAILED DESCRIPTION OF THE INVENTION

Hydrolysate of Lignocellulosic Materials:

The hydrolysate is obtained by heat treatment of all types of lignocellulosic materials in the presence of water or water vapour. This treatment in the presence of water or water vapour allows the preparation of the hydrolysate and of a residue rich in cellulose, and optionally in lignin. Advantageously, this residue is then separated from the hydrolysate, for example via filtration. According to one embodiment, the lignocellulosic materials are chosen from wood, recycled wood, annual plants, agricultural residues, recovered papers and cardboards, and bagasse, preferably wood.

According to one embodiment of the invention, the hydrolysate of lignocellulosic materials is obtained by hydrolysis using water or water vapour to which a weak or strong acid has been added.

The hydrolysate of lignocellulosic materials comprises hemicelluloses in the form of monomers, oligomers or polymers. It also comprises products of degradation of the sugars, such as hydroxymethylfurfural and furfural, and products resulting from the lignin and/or from the products of degradation of the components of the wood (these products can be either in dissolved form or precipitated form). It can also contain organic acids, as well as products from the extractables of the wood.

According to one embodiment of the invention, the hydrolysate of lignocellulosic materials has a total concentration of sugars between 15 and 45 g/L.

Oxidant

The method comprises at least one step of oxidation with at least oxidant. According to one embodiment, the quantity of oxidant used is between 1 and 100% by weight with respect to the quantity of lignin (precipitated and dissolved), of furfural, of hydroxymethylfurfural and of the coloured compounds present in the hydrolysate.

According to a specific embodiment of the invention, the oxidant is chosen from hydrogen peroxide, ozone, oxygen, the peracids, the free radicals and the chlorinated oxidants such as chlorine dioxide.

Preferably, the oxidant is chosen from hydrogen peroxide and ozone.

According to one embodiment of the invention, the oxidation is carried out with a combination of two oxidants, for example hydrogen peroxide and ozone or hydrogen peroxide and oxygen.

Methodology

According to a preferred embodiment of the invention, the method according to the invention comprises the following steps:
implementation of a hydrolysate of lignocellulosic materials;
optionally, adjustment of the pH, in order to obtain a pH between 3.5 and 12;
placement of the hydrolysate in contact with at least one oxidant;
optionally, heating of the reaction medium to a temperature less than or equal to [in ° C. and in increasing order of preference]: 120; 110; 100; 90; 80; 70; for example between 20 and 100° C.;
recovery of a mixture of monosaccharides and/or of oligosaccharides and/or of polysaccharides containing less furfural and hydroxymethylfurfural and in which the molecular weight of the polymers of lignin and/or of products of degradation has been reduced;
optionally, neutralisation of the residual oxidant.

According to one embodiment, the method according to the invention comprises the following steps:
heat treatment of the lignocellulosic materials in the presence of water or water vapour in order to obtain a hydrolysate of lignocellulosic materials and a residue rich in cellulose, and optionally in lignin;
separation of the hydrolysate of lignocellulosic materials from the residue rich in cellulose, for example via filtration;
optionally, adjustment of the pH of the hydrolysate, in order to obtain a pH between 3.5 and 12;
placement of the hydrolysate in contact with at least one oxidant;
optionally, heating of the reaction medium, to a temperature less than or equal to [in ° C. and in increasing order of preference]: 120; 110; 100; 90; 80; 70; for example between 20 and 100° C.;
recovery of a mixture of monosaccharides and/or of oligosaccharides and/or of polysaccharides containing less furfural and hydroxymethylfurfural and in which the molecular weight of the polymers of lignin and/or of products of degradation has been reduced;
optionally, neutralisation of the residual oxidant.

Advantageously, the step of oxidation is carried out at a pH between 1 and 13, preferably between 3.5 and 11.

According to one embodiment of the invention, when the oxidant is hydrogen peroxide, the reaction is carried out at a pH between 8 and 12.

Advantageously, the pH is adjusted via the addition of soda to the reaction medium.

According to another embodiment of the invention, when the oxidant is ozone, the reaction is carried out at a pH between 2 and 5.

According to a specific embodiment of the invention, the pH is not modified and the step of oxidation is carried out at the pH of the hydrolysate of lignocellulosic materials, that is to say, between 3 and 3.5.

Preferably, the step of oxidation is carried out at a temperature less than or equal to [in ° C. and in increasing order of preference]: 120; 110; 100; 90; 80; 70; for example between 20 and 100° C. The temperature at which the step of oxidation is carried out is one of multiple important parameters. Indeed, it is preferable that this temperature not be too high in order to prevent degradation of the monomer and oligomer sugars contained in the hydrolysate.

According to one embodiment of the invention, when the oxidant is ozone, the reaction is carried out at ambient temperature.

According to a specific embodiment of the invention, the oxidising treatment lasts between several minutes and 4 hours, preferably between several minutes and 2 hours.

The oxidation reaction is for example carried out at atmospheric pressure. The oxidation reaction can also be carried out under pressure. For example, the reaction can be carried out at a pressure of less than 10 bar or between 5 and 10 bar.

Mixture of Monosaccharides and/or of Oligosaccharides and/or of Polysaccharides

This method allows a mixture of monosaccharides and/or of oligosaccharides and/or of polysaccharides optionally comprising residual traces of oxidant and of the organic acids to be obtained.

This method allows a mixture of monosaccharides and/or of oligosaccharides and/or of polysaccharides to be obtained comprising polymers from the lignin, extractables from the wood and/or products of degradation of the hemicelluloses, having a mass molecular in weight and/or in number reduced by at least 10%, preferably 25% and even more preferably 50% with respect to these same polymers contained in the hydrolysate of lignocellulosic materials.

This method allows a mixture of monosaccharides and/or of oligosaccharides and/or of polysaccharides to be obtained comprising a quantity of furfural and of hydroxymethylfurfural reduced by at least 10%, preferably 25% and even more preferably 50%, with respect to the quantity of furfural and of hydroxymethylfurfural present in the hydrolysate of lignocellulosic materials.

Uses:

The mixture of monosaccharides and/or of oligosaccharides and/or of polysaccharides obtained after treatment can be used industrially and the sugars contained in this mixture can be separated and purified.

### Examples

The hydrolysate of lignocellulosic materials and the mixture of monosaccharides and/or of oligosaccharides and/or of polysaccharides are analysed via high-pressure high-performance anion-exchange chromatography coupled with a pulsed amperometric detector (HPAEC-PAD, for High Performance Anion Exchange Chromatography-Pulsed Amperometric Detection). The chromatography system is a Dionex ICS 5000 model, consisting of a guard column (50 mm×4 mm), a CarboPac PA10 column (250 mm×4 mm) and a pulsed amperometric detector consisting of a reference electrode and a gold electrode.

1) Preparation of the Hydrolysate of Lignocellulosic Materials.

The hydrolysate of lignocellulosic materials was obtained via autohydrolysis of 400 g (expressed in dry matter) of wood chips of broad-leaved trees in the following conditions:
Liquid/Dry wood Ratio=2
Temperature: 170° C.
Time to reach temperature: 30 min
Time at temperature: 120 min

Cooling time (to 50° C.): 30 min

The hydrolysate was then separated from the chips via filtration.

The sugar composition of the autohydrolysate obtained is presented in table 1. Its pH is 3.5.

TABLE 1
sugar composition of the hydrolysate of lignocellulosic materials

| | Quantity (g/L) | | |
|---|---|---|---|
| Sugar | Monomers | Oligomers | Total |
| Arabinose | 0.6 | 0.0 | 0.6 |
| Galactose | 1.7 | 0.4 | 2.1 |
| Glucose | 1.2 | 1.6 | 2.8 |
| Xylose | 12.9 | 5.9 | 18.8 |
| Mannose | 1.3 | 2.1 | 3.4 |
| Total Pentoses | 13.5 | 5.9 | 19.1 |
| Total Hexoses | 4.2 | 4.1 | 8.3 |
| Total sugars | 17.7 | 10.0 | 27.7 |

The other components of the hydrolysate of lignocellulosic materials obtained are the following:
the quantity of hydroxymethylfurfural (HMF) is 0.6 g/L,
the quantity of furfural is 6.3 g/L.
the quantity of soluble lignin and coloured compounds is 8.6 g/L,
the quantity of insoluble lignin and coloured compounds is 5.6 g/L.

The total concentration of other components is therefore 21.1 g/L.

2) Treatment of the Hydrolysate with Hydrogen Peroxide

A volume of 50 ml of the hydrolysate obtained previously is used as such. The pH of the hydrolysate is first adjusted if necessary with soda and then the hydrolysate is placed in contact with a diluted commercially-available solution of hydrogen peroxide. The concentration of hydrogen peroxide in the hydrolysate is set to 16 g/L. Then, the solution is heated at the desired temperature for a given time. The conditions are summarised in table 2.

TABLE 2
Conditions of the various tests carried out with hydrogen peroxide

| Test | initial | Temperature | Time |
|---|---|---|---|
| n° | pH | (° C.) | (h) |
| 1 | 3.5 | 70 | 2 |
| 2 | 10 | 70 | 2 |
| 3 | 3.5 | 80 | 4 |
| 4 | 12 | 80 | 4 |
| 5 | 12 | 90 | 4 |

The mixture of monosaccharides and/or of oligosaccharides and/or of polysaccharides obtained after treatment is analysed.

Table 3 summarises the effects of the oxidising treatment on the averages of the masses molecular in number (Mn) and in weight (Mw) of the lignin and of the insoluble and soluble coloured compounds. According to the treatment conditions, a decrease in the mass molecular in number that can reach 79% and in the mass molecular in weight up to 76% is observed.

TABLE 3
Change in the masses molecular in number (Mn) and
in weight (Mw) of the lignin and of the insoluble and
soluble coloured materials according to the various treatments.

| Treatment | Effect on the insoluble | | | |
|---|---|---|---|---|
| (test | lignin and coloured | | Effect on the soluble lignin | |
| number, cf | compounds | | and coloured compounds | |
| table 2) | ΔMn/Mn, % | ΔMw/Mw, % | ΔMn/Mn, % | ΔMw/Mw, % |
| 1 | 0 | 27 | 32 | 13 |
| 2 | 0 | 29 | 42 | 50 |
| 3 | 0 | 38 | 52 | 18 |
| 4 | 27 | 63 | 77 | 73 |
| 5 | 27 | 52 | 79 | 76 |

The effect of the treatments with hydrogen peroxide on the furfural and on the HMF was measured (FIG. 1). The concentration of furfural and of HMF is reduced by more than 50% in the mildest operating conditions (pH 3.5, 70° C., 2h and pH 10, 70° C., 2 hr). In the harshest conditions, the concentration of furfural is practically zero. The concentrations of HMF, already low at the beginning, can be divided by 3.

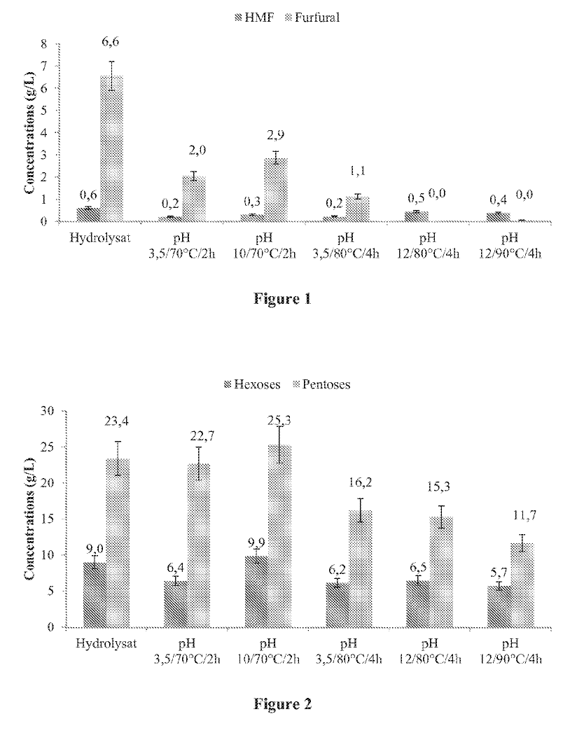

The sugar composition was verified after treatment. The results presented in FIG. 2 show that in the two mildest conditions (pH 3.5, 70° C., 2 hrs, and pH 10, 70° C., 2 hr), no significant impact on the pentoses or on the hexoses is observed for the test in a basic medium.

3) Treatment of the Hydrolysate with Ozone

The treatments with ozone were carried out at the pH of the hydrolysate, or approximately 3-3.5, at ambient temperature, with ozone levels increasing between 4 and 16 g of ozone introduced/litre of hydrolysate.

The same analyses as for the treatment with hydrogen peroxide were carried out. Table 4 shows that the treatment with ozone is effective starting with the small levels of ozone added.

TABLE 4
Change in the masses molecular in number (Mn) and in weight (Mw)
of the lignin and of the insoluble and soluble coloured materials
according to the various treatments with ozone.

| Treatment | | | | |
|---|---|---|---|---|
| with | | | | |
| ozone | Effect on the | | | |
| Level of | insoluble lignin and | | Effect on the soluble lignin | |
| ozone, | coloured compounds | | and coloured compounds | |
| g/L | ΔMn/Mn, % | ΔMw/Mw, % | ΔMn/Mn, % | ΔMw/Mw, % |
| 4 | 0 | 50 | 62 | 46 |
| 8 | 0 | 35 | 64 | 44 |
| 12 | 0 | 30 | 59 | 43 |
| 16 | 0 | 67 | 60 | 40 |

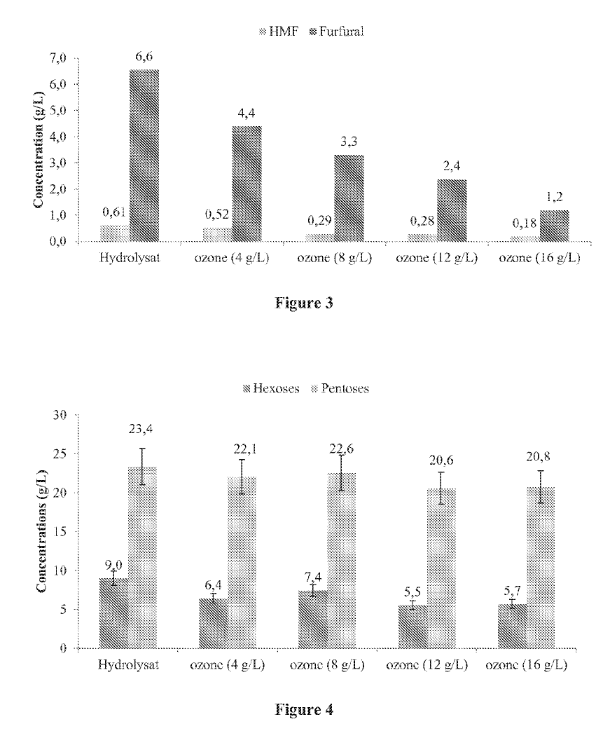

The ozone also leads to a significant reduction in the quantities of furfural and HMF (FIG. 3).

With regard to the impact on the sugar concentrations (FIG. 4), it is interesting to note that the ozone does not have an effect on the pentoses until 8 g/L, and then the concentrations only go down very slightly, which is surprising given that ozone is known for oxidising sugars and degrading them.

### KEY TO THE FIGURES

FIG. 1

| | |
|---|---|
| Hydrolysat | Hydrolysate |

FIG. 2

| | |
|---|---|
| Hydrolysat | Hydrolysate |

FIG. 3

| | |
|---|---|
| Hydrolysat | Hydrolysate |

FIG. 4

| | |
|---|---|
| Hydrolysat | Hydrolysate |

1. Method for preparing a mixture of monosaccharides and/or of oligosaccharides and/or of polysaccharides, via purification of a hydrolysate of lignocellulosic materials, said hydrolysate comprising hemicelluloses in the form of monomers and of oligomers, and optionally of polymers, characterised in that the method comprises at least one step of oxidation of said hydrolysate with at least one oxidant.

2. Method according to claim 1, wherein the lignocellulosic materials are chosen from wood, recycled wood, annual plants, agricultural residues and recovered papers and cardboards.

3. Method according to claim 1, wherein said hydrolysate also comprises products of degradation of the sugars, extractables of the wood and polymers from the lignin and/or from the products of degradation of the sugars.

4. Method according claim 1, wherein the quantity of oxidant used is between 1 and 100% by weight with respect to the quantity of lignin (precipitated and dissolved), of furfural, of hydroxymethylfurfural and of coloured compounds present in the hydrolysate.

5. Method according to claim 1, wherein the oxidant is chosen from hydrogen peroxide, ozone, the peracids, the free radicals and the chlorinated oxidants.

6. Method according to claim 1, wherein the oxidant is chosen from ozone and hydrogen peroxide.

7. Method according to claim 1, wherein the step of oxidation is carried out at a pH between 1 and 13.

8. Method according to claim 1, wherein the step of oxidation is carried out at a temperature less than or equal to 120° C.

9. Method according to claim 1, comprising the following steps:
implementation of a hydrolysate of lignocellulosic materials;
placement of the hydrolysate in contact with at least one oxidant;
recovery of a mixture of monosaccharides and/or of oligosaccharides and/or of polysaccharides containing less furfural and hydroxymethylfurfural and in which the molecular weight of the polymers of lignin and/or of products of degradation has been reduced;

10. Method according to claim 1, comprising the following steps:
heat treatment of the lignocellulosic materials in the presence of water or water vapour in order to obtain a hydrolysate of lignocellulosic materials and a residue rich in cellulose, and optionally in lignin;
separation of the hydrolysate of lignocellulosic materials from the residue rich in cellulose, for example via filtration;
adjustment of the pH of the hydrolysate, in order to obtain a pH between 3.5 and 12;
placement of the hydrolysate in contact with at least one oxidant;
heating of the reaction medium, to a temperature less than or equal to 120° C.;
recovery of a mixture of monosaccharides and/or of oligosaccharides and/or of polysaccharides containing less furfural and hydroxymethylfurfural and in which the molecular weight of the polymers of lignin and/or of products of degradation has been reduced;
neutralisation of the residual oxidant.

11. Method according to claim 1, wherein the reaction is carried out at the pH of the hydrolysate of lignocellulosic materials.

12. Mixture of monosaccharides and/or of oligosaccharides and/or of polysaccharides suitable for being obtained by purification of a hydrolysate of lignocellulosic materials according to claim 1, wherein the mixture contains polymers from the lignin, extractables from the wood and/or products of degradation of the sugars, having a mass molecular in weight and/or in number reduced by at least 10% with respect to the polymers contained in the hydrolysate of lignocellulosic materials.

13. Mixture of monosaccharides and/or of oligosaccharides and/or of polysaccharides according to claim 12, wherein the mixture contains a quantity of furfural and of hydroxymethylfurfural reduced by at least 10% with respect to the quantity of furfural and of hydroxymethylfurfural present in the hydrolysate of lignocellulosic materials.

14. Method according to claim 7, wherein the step of oxidation is carried out at a pH between 3.5 and 11.

15. Method according to claim 8, wherein the step of oxidation is carried out at a temperature less than or equal to at least one of 110° C., 100° C., 90° C., 80° C., or 70° C.

16. Method according to claim 9, further comprising:
adjusting pH in order to obtain a pH between 3.5 and 12.

17. Method according to claim 9, further comprising:
heating of the reaction medium, to a temperature less than or equal to 120° C.

18. Method according to claim 9, further comprising:
neutralizing the residual oxidant.

19. Mixture of monosaccharides and/or of oligosaccharides and/or of polysaccharides according to claim 12, wherein the mixture contains polymers from the lignin, extractables from the wood and/or products of degradation of the sugars, having a mass molecular in weight and/or in number reduced by at least 25% with respect to the polymers contained in the hydrolysate of lignocellulosic materials.

20. Mixture of monosaccharides and/or of oligosaccharides and/or of polysaccharides according to claim 12, wherein the mixture contains polymers from the lignin, extractables from the wood and/or products of degradation of the sugars, having a mass molecular in weight and/or in number reduced by at least 50% with respect to the polymers contained in the hydrolysate of lignocellulosic materials.
